(19) United States
(12) Patent Application Publication
(10) Pub. No.: US 2014/0142529 A1
(43) Pub. Date: May 22, 2014

(54) DIAPER

(71) Applicant: TAIWAN PAIHO LIMITED, Chang Hua Hsien (TW)

(72) Inventor: Allen CHENG, Chang Hua Hsien (TW)

(73) Assignee: Taiwan Paiho Limited, Chang Hua Hsien (TW)

(21) Appl. No.: 13/767,688

(22) Filed: Feb. 14, 2013

(30) Foreign Application Priority Data
Nov. 19, 2012 (TW) 101222351

(51) Int. Cl. A61F 13/49; A61F 13/15

(52) U.S. Cl. CPC A61F 13/49011; A61F 13/15699
USPC 604/385.3; 156/73.1

(57) ABSTRACT

A diaper comprising: a front waist portion which has a front waist end with a front waist elastic member, being provided with Velcro floss surface; a rear waist portion which has a rear waist end with a rear waist elastic member being provided with Velcro hook surfaces, wherein the Velcro hook surfaces and the Velcro floss surface are engageable for joining the front waist portion and rear waist portion; and a crotch portion which has a front crotch end and a rear crotch end, wherein the crotch portion joined to the front waist portion and the rear waist portion in a laminated manner, respectively, thus forming accommodating spaces at the joining locations. The crotch portion has thigh opening edges with elastic members forming accommodating spaces. Said accommodating spaces are for preventing the excrement from spilling out of the front, back, left and right sides of the diaper.

### BACKGROUND OF THE INVENTION

1. Technical Field

The present invention relates to a diaper, and in particular to a disposable diaper which can be discarded after use.

2. Description of the Prior Art

For a long time, disposable diapers have been widely used due to the convenience of being discarded after use. Disposable diapers for newborn baby or toddlers available on the market generally include two types: (1) open diapers, and (2) pull-up diapers, which are suitable for infant child at different age levels according to their different usage.

Open diapers are suitable for newborns or infants with limited mobility. The usage thereof is to change a diaper as an infant is in his lying posture, and then correctly wrap the waist and hip of the infant with the open diaper. Therefore, in order to complete the process of putting on the diaper. It is necessary to make the waist area of the open diaper sufficiently engaged and fixed by way of adhesion or Velcro fastening.

Pull-up diapers are suitable for toddlers that are learning to walk or have already had mobility. The usage of pull-up diapers is similar to wearing pants, i.e., a pull-up diaper can be changed as a toddler is standing or lying. In order to prevent the diaper from falling off during the toddler is running, jumping or exercising, the waist portion of a pull-up diaper is often provided with designs having excellent elasticity and easy-to-wear characteristics, such as elastic webbings, elastic tapes or other elastic fibrous materials. Since the way of putting on a pull-up diaper is similar to that of putting on pants, pull-up diapers are often used for training toddlers to wear pants. As a result, such diapers are also called “training pants”.

Regarding the fabrication of diapers, the design and manufacturing process for open diapers is to cut the base material to be H-shaped or T-shaped. Subsequently, the diaper base material, an absorbent member for absorbing excrement, an anti-leakage member for preventing the excrement from leaking out of the thigh openings of the diaper around the crotch portion, an elastic member for providing elasticity, the adhesion members or Velcro members are joined together by sewing or adhering. On the other hand, since pull-up diapers have a stereo configuration, the fabrication thereof is more complicated, and a higher technical level of manufacturing is also required, and thus higher resulting higher manufacturing cost. Therefore, one objective of the present invention is to retain the advantages of both open diapers and pull-up diapers, while simplify the manufacturing processes, and effectively reduce the manufacturing cost.

### SUMMARY OF THE INVENTION

Therefore, for the problems and objectives above, in order to combine the advantages of open diapers and pull-up diapers and develop a diaper fabricating process of high efficiency and low cost, the present invention provides a disposable diaper which is substantially elongated-shaped and has an anterior-posterior direction along its length direction and a lateral direction along its width direction, comprising: a front waist portion having a front waist end and a front waist-crotch area along the anterior-posterior direction, the front waist end being provided with a front waist elastic member along the lateral direction, the front waist end being elastically contractible through the front waist elastic member, and the front waist portion being provided with at least one Velcro floss surface; a rear waist portion having a rear waist end and a rear waist-crotch area, the rear waist end being provided with a rear waist elastic member along the lateral direction, the rear waist end being elastically contractible through the rear waist elastic member, the rear waist potion being provided with a plurality of Velcro hook surfaces disposed on both sides of the lateral direction corresponding to the Velcro floss surface, the Velcro hook surfaces and the Velcro floss surface being engageable after contacting each other, the front waist portion and the rear waist portion being joined through the Velcro hook surfaces and the Velcro floss surface; and a crotch portion provided with an absorbent member for absorbing excrement, the crotch portion having a front crotch end and a rear crotch end along the anterior-posterior direction, the front crotch end being joined to the front waist-crotch area of the front waist portion in a laminated manner, wherein the front crotch end and the front waist-crotch area form a first accommodating space after the joining for preventing the excrement from spilling out of the front waist portion; the rear crotch end being joined to the rear waist-crotch area of the rear waist portion in a laminated manner, wherein the rear crotch end and the rear waist-crotch area form a second accommodating space after the joining for preventing the excrement from spilling out of the rear waist portion, wherein two sides of the crotch portion that are not joined to the front waist-crotch area or the rear waist-crotch area have a thigh opening edge, respectively, the thigh opening edges extend along the anterior-posterior direction, each of the thigh opening edges is provided with a crotch portion elastic member enabling the thigh opening edges to be elastically contractible, and each of the thigh opening edges is provided with a third accommodating space formed along the anterior-posterior direction for preventing the excrement from spilling out of the both sides of the crotch potion.

Based on the abovementioned spirit of the present invention, it is provided a disposable diaper which is substantially elongated-shaped and has an anterior-posterior direction along its length direction and a lateral direction along its width direction, comprising: a first waist portion having a first waist end and a first waist-crotch area along the anterior-posterior direction, the first waist end being provided with a first waist elastic member along the lateral direction, the first waist end being elastically contractible through the first waist elastic member, and the first waist portion being provided with at least one Velcro floss surface; a second waist portion having a second waist end and a second waist-crotch area along the anterior-posterior direction, the second waist end being provided with a second waist elastic member along the lateral direction, the second waist end being elastically contractible through the second waist elastic member, the second waist potion being provided with a plurality of Velcro hook surfaces disposed on both sides of the lateral direction corresponding to the Velcro floss surface, the Velcro hook surfaces and the Velcro floss surface being engageable after contacting each other, the first waist portion and the second waist portion being joined through the Velcro hook surfaces and the Velcro floss surface; and a crotch portion provided with an absorbent member for absorbing excrement, the crotch portion having a first crotch end and a second crotch end along the anterior-posterior direction, the first crotch end being joined to the first waist-crotch area of the first waist portion in a laminated manner, wherein the first crotch end and the first waist-crotch area form a first accommodating space after the joining for preventing the excrement from spilling out of the first waist portion, wherein two sides of the crotch portion that are not joined to the first waist-crotch area or the second waist-crotch area have a thigh opening edge, respectively, the thigh opening edges extend along the anterior-posterior direction, each of the thigh opening edges is provided with a crotch portion elastic member enabling the thigh opening edges to be elastically contractible, and each of the thigh opening edges is provided with a second accommodating space formed along the anterior-posterior direction for preventing the excrement from spilling out of the both sides of the crotch potion.

Additionally, in the abovementioned diaper of the present invention, the front waist portion and the rear waist portion are made of a nonwoven fabric which is elastically contractible.

Further, in the abovementioned diaper, the nonwoven fabric of the front waist portion is joined to the front crotch end in a laminated manner, enabling the joined front crotch end and front waist-crotch area to form the first accommodating space; the nonwoven fabric of the rear waist portion is joined to the rear crotch end in a laminated manner, enabling the joined rear crotch end and rear waist-crotch area to form the second accommodating space.

In addition, the absorbent member of the abovementioned diapers has a layered structure, and the layered structure includes at least a permeable layer, a liquid absorbing-holding layer, and an impermeable layer.

In addition, in the abovementioned diapers, the joining of the respective portions is performed by ultrasonic fusion joining or adhesive joining for a firm attachment.

The spirit of the present invention described above includes but is not limited to: combining the known advantages of open diapers and pull-up diapers, increasing the functionality of using a diaper, simplifying fabricating processes and lowering fabricating cost. The present invention provides a disposable diaper provided with Velcro for joining, the diaper is flat before being used, which includes the advantage of an open diaper. On the other hand, since the diaper is provided with elastic members at its waist portion, after the diaper is joined with Velcro, it exhibits a state of short pants with elastically contractible waist portion and a pair of thigh openings, which allows toddlers to wear the diaper while he is standing, and the diaper can be used for training toddlers to put on or take off pants. Thus the diaper has the advantage of a pull-up diaper as well. As a result, the diaper of the present invention is widely suitable for infants of various ages, ranging from newborns to infants learning to walk, and toddlers full of activity.

Moreover, for the diaper provided by the present invention, with its design of portion-dividing and portion-joining, not only a diaper of excellent productivity, improved yield and lowered cost can be provided, but also pocket-like structures for preventing leakage of excrement can be formed when joining the respective portions of the diaper in a laminated manner without any additional process, and resulting in the improved convenience and functionality of the diaper of the present invention.

### BRIEF DESCRIPTION OF THE DRAWINGS

FIG. 1A is a schematic top view showing the crotch portion, front waist portion and rear waist portion of a diaper according to a first embodiment of the present invention.

FIG. 1B is a schematic top view showing the crotch portion, front waist portion and rear waist portion of a diaper according to a second embodiment of the present invention.

FIG. 2 is a back side perspective view showing the crotch portion, front waist portion and rear waist portion of a diaper according to the first embodiment of the present invention.

FIG. 3 is a front perspective view showing the crotch portion, front waist portion and rear waist portion of a diaper according to the first embodiment of the present invention.

FIG. 4 is a schematic top view showing the crotch portion, front waist portion and rear waist portion of a diaper according to the third embodiment of the present invention.

FIG. 5 is a front perspective view showing the crotch portion, front waist portion and rear waist portion of a diaper according to the third embodiment of the present invention.

### DETAILED DESCRIPTION OF THE PREFERRED EMBODIMENTS

In order to make the objectives, technical features and advantages of the present invention to be better understood and practiced by those skilled in the art, preferred embodiments are set forth together with accompanying drawings for further describing the technical features and implementing measures of the present invention. However, the embodiments below are not used to limit the present invention.

A diaper is substantially elongated-shaped when it is spread flat, and the longitudinal direction thereof corresponds to the anterior-posterior direction of the wearer when wearing the diaper (“front” means the abdomen of the wearer, and “rear” means the back of the wearer), so herein after, such direction is called “anterior-posterior direction” and denoted by X. The width direction of the diaper corresponds to the side-to-side direction of the wearer when wearing the diaper, so hereinafter, such direction is called “lateral direction” and denoted by Y. The diaper forms a three dimensional shape of a pair of short pants when fully bonded. At this time, the direction corresponds to the height direction of the standing wearer is called “thickness direction” and denoted by Z.

Based on corresponding positions of the diaper contacting the wearer, the diaper of the present invention comprises a front waist portion, a crotch portion and a rear waist portion. These portions may be three separated single pieces that are firmly engaged by following a predetermined sequence. As an alternative, these portions may be configured to be one single piece in which the front waist portion and the crotch portion are joined or integrally formed and another single piece of the rear waist portion, which are firmly engaged. Similarly, these portions may be configured to be one single piece in which the rear waist portion and the crotch portion are joined or integrally formed and another single piece of the front waist portion, which are firmly engaged. All of these configurations above can construct the present invention. The terms “front” and “rear” are used here for the convenience of explanation. Without distinguishing between the front and rear sides of the diaper of the present invention, the abovementioned front waist portion can be referred to as a first waist portion, while the abovementioned rear waist portion is referred to as a second waist portion. Alternatively, the abovementioned rear waist portion can be referred to as a first waist portion, while the abovementioned front waist portion is referred to as a second waist portion. Thus, the terms “front” and “rear” in the following description are not intended to limit the embodiments of the present invention.

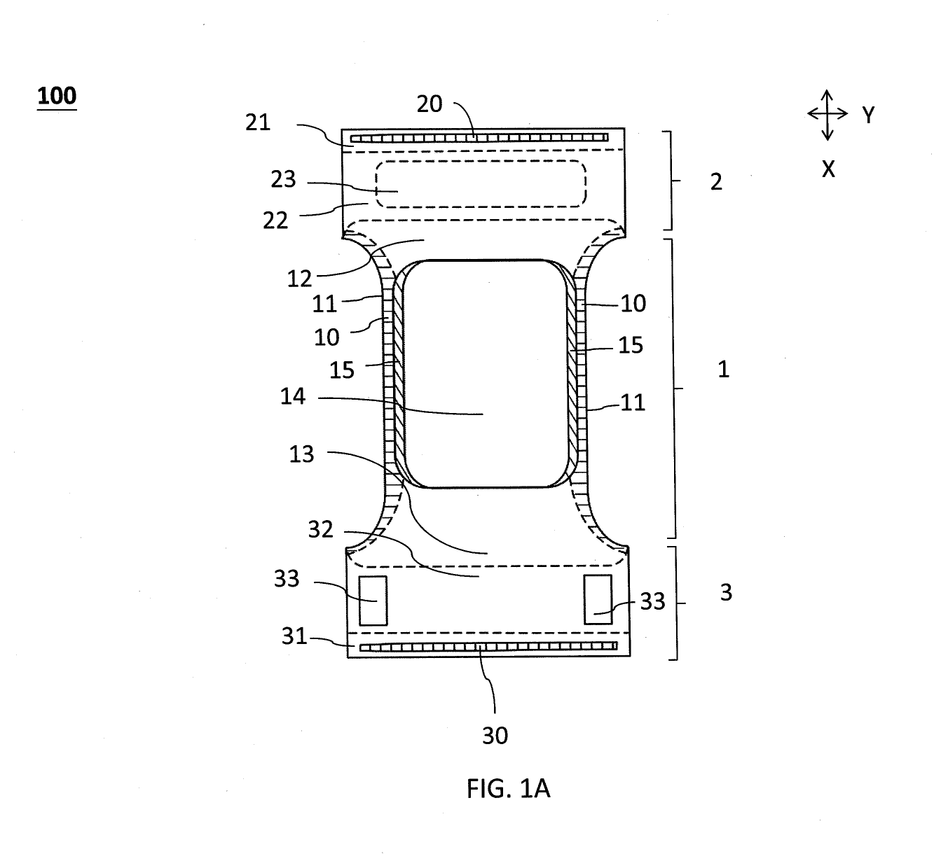

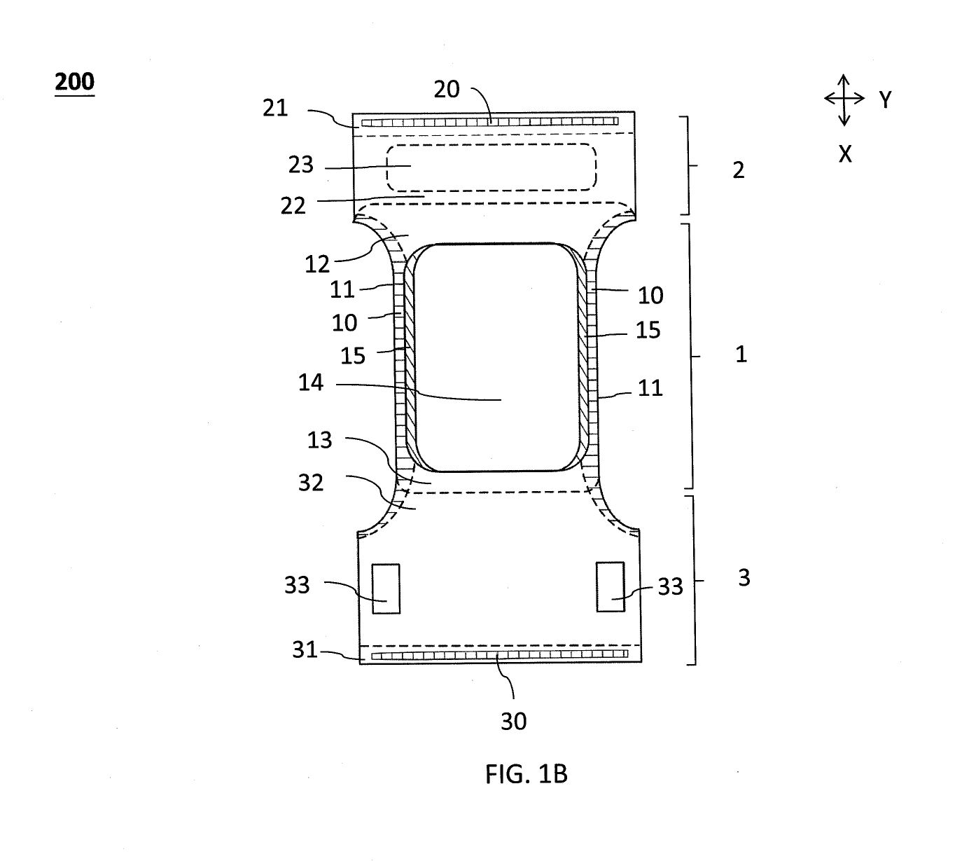

First, a first embodiment is used to describing the present invention. Please refer to FIG. 1A which is a schematic top view showing the engagement among the respective portions and elements of the diaper when the diaper 100 according to a first embodiment of the present invention is spread flat. The diaper 100 of this embodiment is substantially elongated-shaped, with an anterior-posterior direction X along its longitudinal orientation and a lateral direction Y along its width orientation. The diaper 100 comprises three single pieces: a front waist portion 2, a crotch portion 1, and a rear waist portion 3, wherein the front waist portion 2 and the rear waist portion 3 have substantially symmetric shapes.

The front waist portion 2 of the diaper 100 further includes, along the anterior-posterior direction X, a front waist end 21 and a front waist-crotch area 22 that is in connection with the crotch portion 1. A front waist elastic member 20 is provided at the front waist end 21 along the lateral direction Y, such that the front waist end 21 is elastically contractible. The front waist elastic member 20 of the present invention is generally referred to a device or structure that can make the front waist end 21 elastically contractible. In order to provide the function of the front waist elastic member 20, for example, this front waist elastic member 20 may be selected from elastic webbings, elastic tapes or elastic fibrous materials and then firmly mounted to the front waist end 21; or the front waist elastic member 20 may be formed by using and corrugating the material of the front waist portion 2 (e.g. elastic nonwoven fabric or elastic cloth) having an elastic characteristics for constructing an elastic structure.

The rear waist portion 3 of the diaper 100 further includes, along the anterior-posterior direction X, a rear waist end 31 and a rear waist-crotch area 32 that is in connection with the crotch portion 1. A rear waist elastic member 30 is provided at the rear waist end 31 along the lateral direction Y, such that the rear waist end 31 is elastically contractible. The rear waist elastic member 30 of this embodiment is not particularly limited in its selection, and a preferred aspect thereof is similar to the front waist elastic member 20 and will not be described again.

The crotch portion of the diaper 100 of this embodiment further includes, along the anterior-posterior direction X, a front crotch end 12 and a rear crotch end 13. The front crotch end 12 is in connection with the abovementioned front waist-crotch area 22. The crotch portion of the diaper includes an absorbent member 14 for absorbing urine or feces. The absorbent member 14 has a layered structure, which includes therein at least one permeable layer for allowing excrement to enter the absorbent member through this layer rapidly and effectively; a liquid absorbing-holding layer that is preferably a polymer material layer with an excellent liquid absorptivity for absorbing and receiving the excrement, so that the excrement cannot easily to leak back to the permeable layer; and an impermeable layer mainly for preventing the excrement from leaking out of the absorbing-holding layer. However, in addition to the abovementioned design of layers, the layer design in the layered structure can be adjusted according to manufacturing needs.

Besides, the crotch portion 1 is respectively in connection with the front waist-crotch area 22 and rear waist-crotch area 32 on two ends along the anterior-posterior direction X, while each of the two sides thereof that are not in connection with the front waist-crotch area 22 and rear waist-crotch area 32 is provided with a thigh opening edge 11 which extends along the anterior-posterior direction X. Further, the thigh opening edges are respectively provided with crotch elastic members 10 for enabling the thigh opening edges 11 to be elastically contractible.

The crotch elastic members 10 of the present invention are generally referred to a device or structure that can make the thigh opening edges 11 of the diaper 100 elastically contractible. In order to provide the function of the crotch elastic members 10, for example, this crotch elastic members 10 may be selected from elastic webbings or elastic fibrous materials and firmly mounted, or may be formed by using and corrugating the material of the crotch portion 1 (e.g. elastic nonwoven fabric or elastic cloth) having an elastic characteristics for constructing an elastic structure. The elastic contractibility of the crotch elastic members 10 also cause the thigh opening edges 11 of the diaper 100 to form a stereo structure similar to baffles.

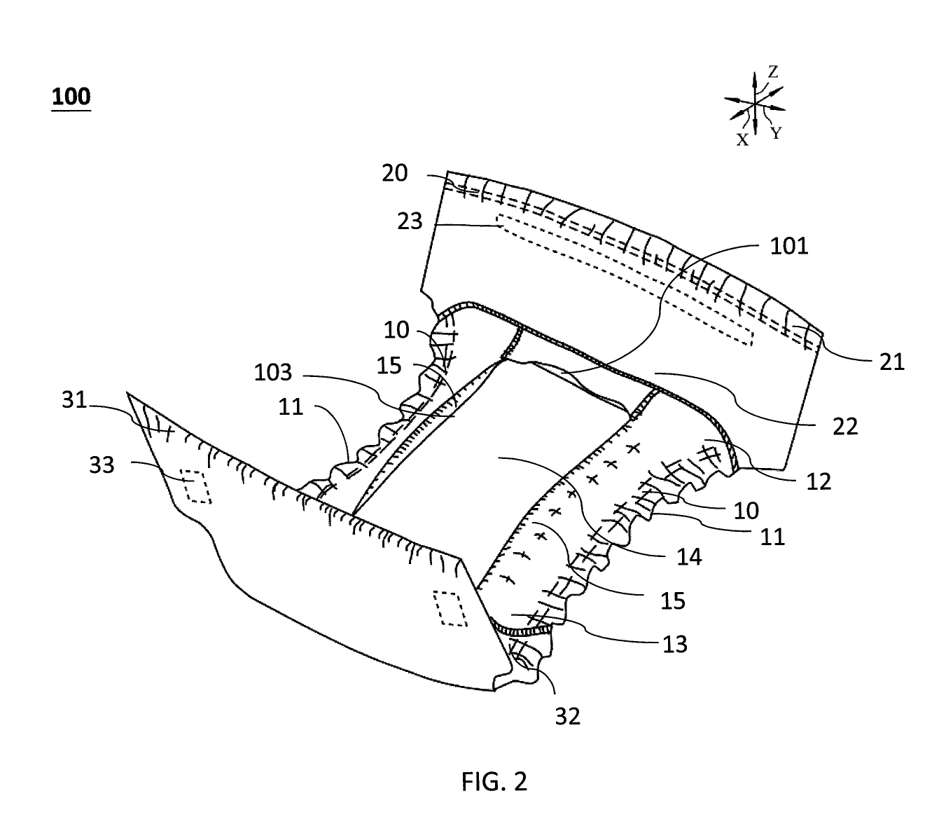

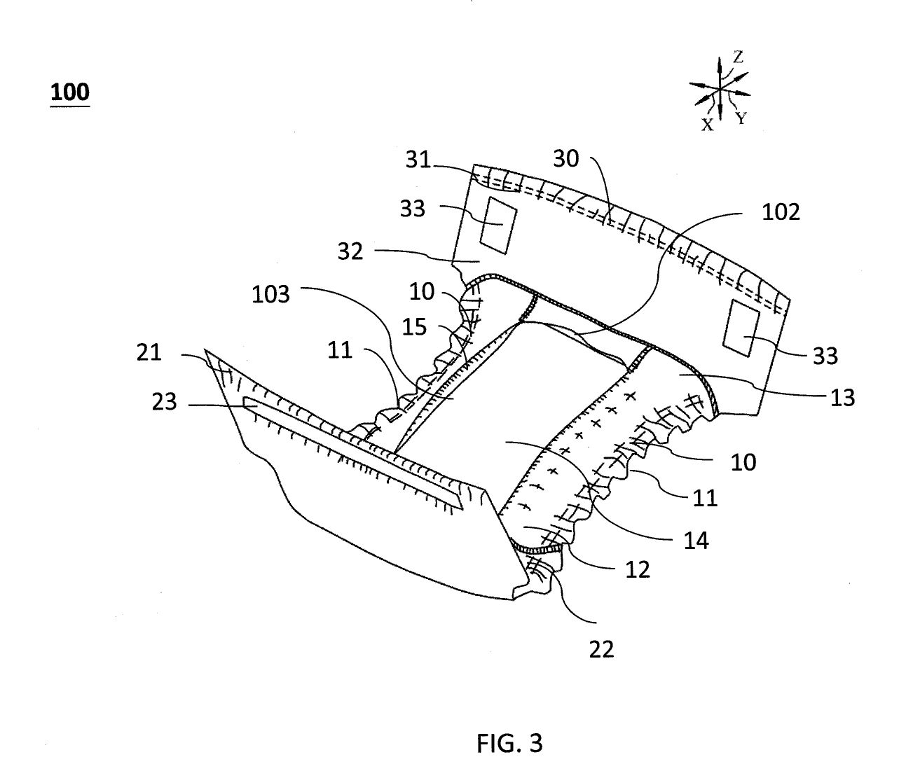

Continue referring to FIG. 2 and FIG. 3, each of the abovementioned thigh opening edges 11 is provided with a left-right accommodating space 103 formed along the anterior-posterior direction X. The left-right accommodating spaces 103 can be formed with the stereo structure established by the elastic contractibility with which the crotch elastic members 10 provide the thigh opening edges 11, or can be formed by additionally disposing an anti-leakage member 15. However, no matter which of the aforementioned ways is taken for forming the left-right accommodating spaces 103, the purpose of the left-right accommodating spaces 103 is to prevent excrement from leaking out of both sides of the crotch portion 1 (i.e., out of the thigh opening edges 11) for achieving an effect of leakage prevention.

The rear waist portion 3 of the diaper 100 of this embodiment further comprises the rear waist end 31 and the rear waist-crotch area 32 along the anterior-posterior direction X, wherein the rear waist-crotch area 32 is in connection with the aforementioned rear crotch end 13.

In the diaper 100 of the first embodiment of the present invention, the material for the front waist portion 2 and rear waist portion 3 can be selected from woven fabrics, polymeric fabrics and nonwoven fabrics. It is preferable that the front waist portion 2 and rear waist portion 3 of the diaper 100 are made of nonwoven fabric with elastic contractibility. More preferably, such nonwoven fabric has excellent air permeability and water repellency, so the fabricated diaper products can have an further improved functionality and satisfy users' demand better.

It should be noted that, for the diaper 100 in the first embodiment of the present invention, the connection of the front crotch end 12 and front waist crotch area 22 in the diaper 100 is made by arranging the material of the front waist-crotch area 22 (such as elastic nonwoven fabric) and the front crotch portion 12 in a laminated manner, wherein the front waist-crotch area 22 is above the front crotch portion 12, so that a space is formed by the front waist-crotch area 22 and front crotch portion 12. For retaining this space, in the following joining process, the joining position of the front waist-crotch area 22 is located near an edge of the material, while the edge of the front waist-crotch area 22 is not sealingly joined to the front crotch end 12. As such, after the front waist-crotch area 22 and the front crotch end 12 are joined, the edge portion of the material of the front waist-crotch area 22 (such as the edge portion of an elastic nonwoven fabric) is still in an open state and formed a pocket-like structure or accommodating space. A specific state thereof is shown in the back side perspective view of the diaper 100 in FIG. 2, where the front waist-crotch area 22 and the front crotch end 12 together form an anterior-posterior accommodating space 101 after being joined for preventing excrement from overflowing out of the front waist portion 2.

Similarly, the connection of the rear crotch end 13 and rear waist-crotch area 32 in the diaper 100 may also be made by arranging the material of the rear waist-crotch area 32 (such as elastic nonwoven fabric) and the rear crotch portion 13 in a laminated manner, wherein the rear waist-crotch area 32 is above the rear crotch portion 13, so that a space is formed by the rear waist-crotch area 32 and rear crotch portion 13. For retaining this space, in the following joining process, the joining position of the rear waist-crotch area 32 is located near an edge of the material, while the edge of the rear waist-crotch area 32 is not sealingly joined to the rear crotch end 13. As such, after the rear waist-crotch area 32 and the rear crotch end 13 are joined, the edge portion of the material of the rear waist-crotch area 32 (such as the edge portion of an elastic nonwoven fabric) is still in an open state and formed a pocket-like structure or accommodating space. A specific state thereof is shown in the front side perspective view of this embodiment in FIG. 3, where the rear waist-crotch area 32 and the rear crotch end 13 together form an anterior-posterior accommodating space 102 after being joined for preventing excrement from overflowing out of the rear waist portion 3.

Therefore, when the abovementioned joining means is used in the fabrication of the diaper 100 of this embodiment, by simply adjusting the joining position or site of the front waist-crotch area 22 and front crotch end 12 or the joining position or site of the rear waist-crotch area 32 and rear crotch end 13, not only the joining can be made, but also a structure preventing leakage of excrement is provided at the same time, without any additionally anti-leakage member disposed or connected. Also, it is not necessary to increase fabricating processes and steps, and thus the manufacturing expense and cost are reduced. Furthermore, based on the requirement of promoting the function of preventing excrement leakage, manufacturers may adjust the style (such as tailored shapes) of material edges of the front waist-crotch area 22 or rear waist-crotch area 32, or further improve the way of joining, so that the anterior-posterior accommodating spaces 101 and 102 of the aforementioned pocket-like structure have better anti-leakage performance.

In addition, the present invention does not limit the abovementioned means for firmly joining, as long as the respective parts of the diaper 100 are firmly joined and difficult to be detached. For example, sewing, adhering, fusion joining, and ultrasonic fusion joining can be taken as the joining method of this embodiment.

Please refer to FIG. 1B, which is a schematic view of a diaper 200 according to the second embodiment of the present invention. The diaper 200 of the second embodiment is only different from the diaper 100 of the first embodiment in the symmetry of the front waist portion 2 and rear waist portion 3, while the other features and technical means are identical to those of the first embodiment. In other words, the front waist portion 2 and rear waist portion 3 of the diaper 100 of the first embodiment are symmetric in their shapes, while the front waist portion 2 and rear waist portion 3 of the diaper 200 of the second embodiment are not symmetric, wherein the length of the front waist portion 2 along the direction X is less than the length of the rear waist portion 3 along the direction X. Thus, for the effect and purpose of the present invention, the symmetry of the front waist portion 2 and rear waist portion 3 of the diaper 100 of the first embodiment enables manufacturers to produce the elements for the front waist portion 2 and rear waist portion 3 with materials of the same shapes, which is beneficial for simplifying the fabricating process (such as simplifying molds for cutting cloth or the like, ratio of material used, etc.) and may reduce the cost as well. The asymmetric characteristics of the front waist portion 2 and rear waist portion 3 of the diaper 200 of the second embodiment is originated from the demand of conveniently using the diaper 200. That is to say, in practical wearing condition of the diaper, the rear waist portion 3 (which wraps the rear waist and hip of an infant) is adjusted to be a little longer than the front waist portion 2 in response to the variation of human (infant) body shape, thereby increasing fitness and comfort for wearing the diaper.

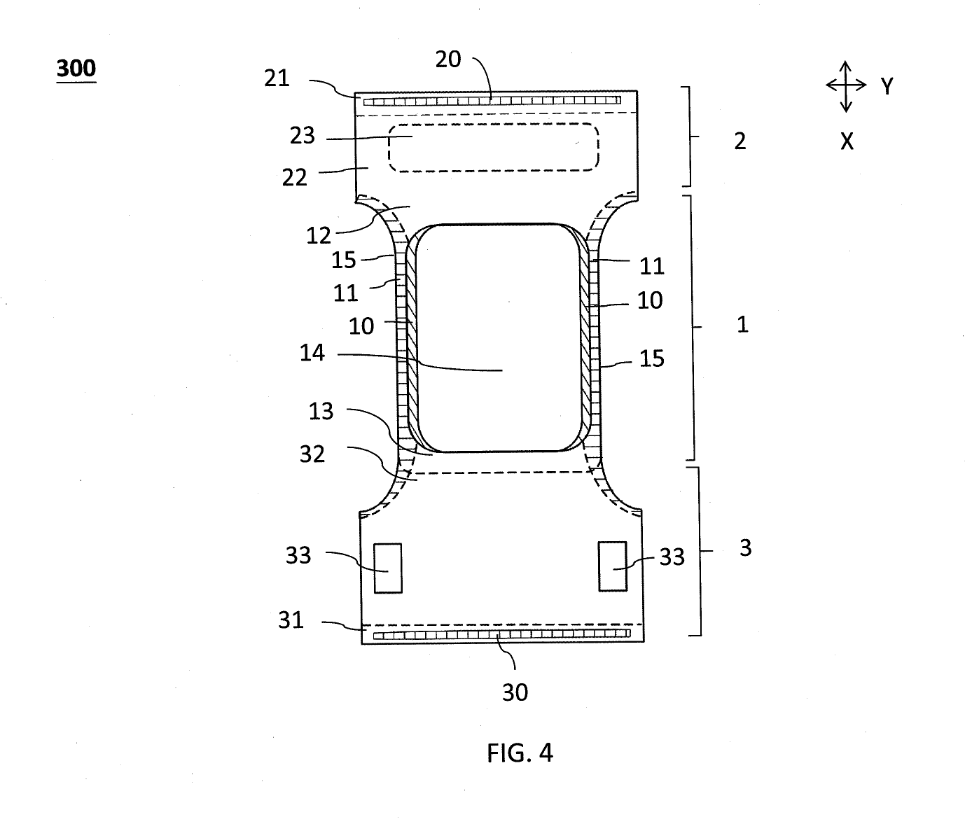

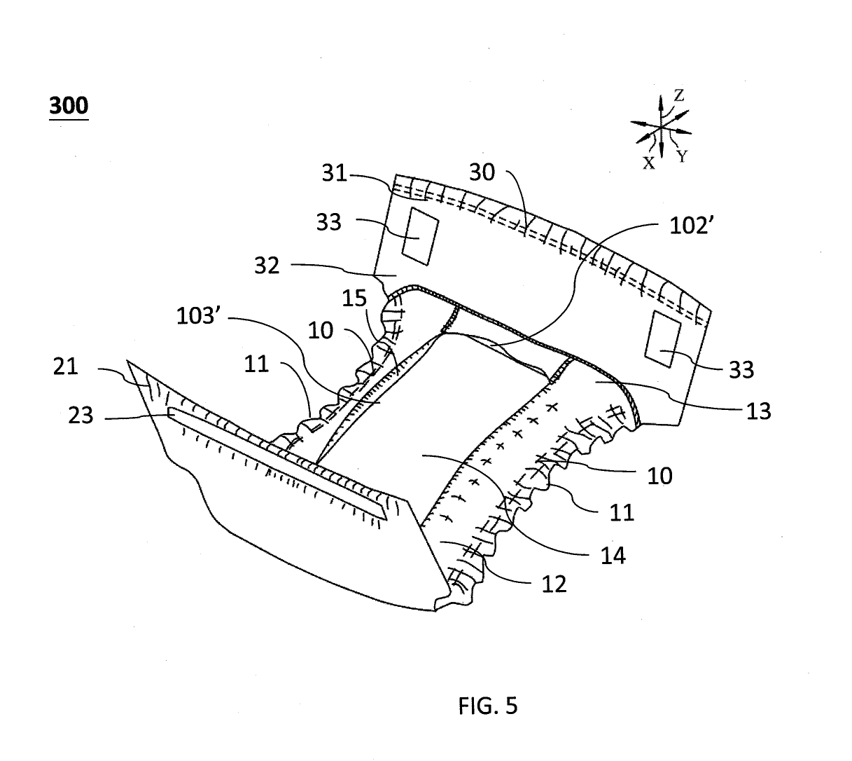

Please continue referring to FIG. 4 and FIG. 5, which are a top view and perspective view of a diaper according to the third embodiment of the present invention. The only difference between the third embodiment and the first embodiment is that, a first waist portion 2 and a crotch portion 1 of the diaper 300 described in the third embodiment have been joined together or become an integrally-formed single member. As a single member of a second waist portion 3 is firmly joined to the aforementioned single member constructed with the first waist portion 2 and crotch portion 1, and an anterior-posterior accommodating space 102′ and a left-right accommodating space 103′ are formed, the diaper described in the third embodiment of the present invention is therefore constructed. Since the way of joining, selection of materials, joining means, configurations of the respective elastic members, and characteristics of the third embodiment shown, when being implemented, are substantially similar to the first embodiment, description therefor will not be made again.

In addition, in order to provide a three-dimensional form for wearing, Velcro is employed in the diaper 100, 200 and 300 of the embodiments of the present invention for joining the front waist portion 2 and rear waist portion 3, so the joined diaper exhibits a stereo short-pants-like style. Such joining means is necessary to be performed with a Velcro set. The Velcro set typically includes a floss surface and a hook surface disposed opposite to the floss surface. As the floss surface contacts the hook surface, the joining means can be achieved, and the floss surface is in mechanical engagement with the hook surface, which can be separated by applying an appropriate force. Further, such joining means is suitable for performing a re-engagement after the separation, so as to achieve an effect of repetitive joining, which is advantageous when the engagement of the first waist portion 2 and second waist portion 3 of the diapers 100, 200 and 300 according to the embodiments of the present invention is adjusted based on the need of users.

Please refer again to FIG. 1A, FIG. 1B, FIG. 2 to FIG. 5. According the implementations above, the joining method for the Velcro of the diapers 100, 200 and 300 according to the embodiments of the present invention is specifically described below. At least one Velcro floss surface 23 is provided on the outer side surface of the front waist portion 2 of the diapers 100, 200 and 300 (i.e. the surface not contacting the wearer's skin when being worn) according to the embodiments of the present invention. On the other hand, a plurality of Velcro hook surfaces 23 are provided on the inner side surface of the rear waist portion 3 (i.e. the surface contacting the wearer's skin when being worn). The aforementioned Velcro hook surfaces 33 are preferably provided on both sides of the rear waist portion along the lateral direction Y, corresponding to the Velcro floss surface 23. Thus, the Velcro hook surface 33 and Velcro floss surface 23 can engage after contacting each other, so that the front waist portion 2 and rear waist portion 3 are joined through the Velcro hook surface 33 and Velcro floss surface 23.

The principle of the above Velcro means is well known by those skilled in the art, where a joining is made by the mechanical engagement of the hooks on the Velcro hook surface 33 and the floss on the Velcro floss surface 23. However, for such principle, the selection of the Velcro floss surface for the present invention is not limited. If the material of the front waist portion 2 of the diaper has a property similar to the floss structure of the Velcro floss surface 23, such material can be used as the Velcro floss surface without disposing an additional Velcro floss surface 23, and the Velcro hook surface 33 may therefore engage the material of the front waist portion 2 of the diaper.

Please refer to the perspective views in FIG. 2, FIG. 3 and FIG. 5, one of the two implementing occasions for the abovementioned Velcro joining means is when the diaper is worn by a newborn or an infant that cannot stand, and the diaper needs to be put on as the infant is in its lying posture. First, the rear waist portion 3 of the flat-spread, unjoined diaper 100, 200 or 300 is placed underneath the rear waist and the hip of the infant. Next, the crotch portion 1 of the diaper 100, 200 or 300 is pulled up along the crotch of the infant, so the crotch portion 1 sufficiently wraps the crotch to receive the excrement of the infant. Next, the front waist portion 2 of the diaper 100, 200 or 300 is made adequately cover the front waist of the infant, and the corresponding position of the Velcro is adjusted according to the body shape of the infant. Then the Velcro floss surface 23 disposed on the front waist portion 2 and Velcro hook surface 33 disposed on the rear waist portion 3 of the diaper 100, 200 or 300 are engaged for fully putting on then diaper.

Another implementing occasion for the abovementioned joining means is when the diaper is worn by en elder toddler being able to stand. In this case, the Velcro floss surface 23 of the front waist portion 2 of the diaper 100, 200 or 300 contacts the Velcro hook surface 33 of the rear waist portion 3. Next, an engagement is completed so the diaper forms a three dimensional shape of a pair of short pants, and then the diaper is worn by the toddler in a manner of wearing a pair of short pants. After the diaper is put on, the engagement thereof can be adjusted using the ability of being repetitively separated and engaged of the Velcro member, which improves the comfort of the wearer.

The abovementioned are the preferred embodiments of the present invention and are not for limiting the present invention. The description above will be understood and implemented by those skilled in the art. Therefore, it is intended that equivalent alterations and modifications made without departing from the spirit disclosed by the present invention are included in the scope of the appended claims.

1. A diaper which is substantially elongated-shaped and has an anterior-posterior direction (X) along its length direction and a lateral direction (Y) along its width direction, comprising:
a front waist portion having a front waist end and a front waist-crotch area along the anterior-posterior direction (X), the front waist end being provided with a front waist elastic member along the lateral direction (Y), the front waist end being elastically contractible through the front waist elastic member, and the front waist portion being provided with at least one Velcro floss surface;
a rear waist portion having a rear waist end and a rear waist-crotch area, the rear waist end being provided with a rear waist elastic member along the lateral direction (Y), the rear waist end being elastically contractible through the rear waist elastic member, the rear waist potion being provided with a plurality of Velcro hook surfaces disposed on both sides of the lateral direction (Y) corresponding to the Velcro floss surface, the Velcro hook surfaces and the Velcro floss surface being engageable after contacting each other, the front waist portion and the rear waist portion being joined through the Velcro hook surfaces and the Velcro floss surface; and
a crotch portion provided with an absorbent member for absorbing excrement, the crotch portion having a front crotch end and a rear crotch end along the anterior-posterior direction (X), the front crotch end being joined to the front waist-crotch area of the front waist portion in a laminated manner, wherein the front crotch end and the front waist-crotch area form a first accommodating space after the joining for preventing the excrement from spilling out of the front waist portion; the rear crotch end being joined to the rear waist-crotch area of the rear waist portion in a laminated manner, wherein the rear crotch end and the rear waist-crotch area form a second accommodating space after the joining for preventing the excrement from spilling out of the rear waist portion,
wherein two sides of the crotch portion that are not joined to the front waist-crotch area or the rear waist-crotch area have a thigh opening edge, respectively, the thigh opening edges extend along the anterior-posterior direction (X), each of the thigh opening edges is provided with a crotch portion elastic member enabling the thigh opening edges to be elastically contractible, and each of the thigh opening edges is provided with a third accommodating space formed along the anterior-posterior direction (X) for preventing the excrement from spilling out of the both sides of the crotch potion.

2. The diaper of claim 1, wherein the front waist portion and the rear waist portion are made of an elastically contractible nonwoven fabric.

3. The diaper of claim 2, wherein the nonwoven fabric of the front waist portion is joined to the front crotch end in a laminated manner, enabling the joined front crotch end and front waist-crotch area to form the first accommodating space.

4. The diaper of claim 2, wherein the nonwoven fabric of the rear waist portion is joined to the rear crotch end in a laminated manner, enabling the joined rear crotch end and rear waist-crotch area to form the second accommodating space.

5. The diaper of claim 1, wherein the absorbent member has a layered structure, the layered structure includes at least a permeable layer, a liquid absorbing-holding layer, and an impermeable layer.

6. A diaper which is substantially elongated-shaped and has an anterior-posterior direction (X) along its length direction and a lateral direction (Y) along its width direction, comprising:
a first waist portion having a first waist end and a first waist-crotch area along the anterior-posterior direction (X), the first waist end being provided with a first waist elastic member along the lateral direction (Y), the first waist end being elastically contractible through the first waist elastic member, and the first waist portion being provided with at least one Velcro floss surface;
a second waist portion having a second waist end and a second waist-crotch area along the anterior-posterior direction (X), the second waist end being provided with a second waist elastic member along the lateral direction (Y), the second waist end being elastically contractible through the second waist elastic member, the second waist potion being provided with a plurality of Velcro hook surfaces disposed on both sides of the lateral direction (Y) corresponding to the Velcro floss surface, the Velcro hook surfaces and the Velcro floss surface being engageable after contacting each other, the first waist portion and the second waist portion being joined through the Velcro hook surfaces and the Velcro floss surface; and
a crotch portion provided with an absorbent member for absorbing excrement, the crotch portion having a first crotch end and a second crotch end along the anterior-posterior direction (X), the first crotch end being joined to the first waist-crotch area of the first waist portion in a laminated manner, wherein the first crotch end and the first waist-crotch area form a first accommodating space after the joining for preventing the excrement from spilling out of the second waist portion,
wherein two sides of the crotch portion that are not joined to the first waist-crotch area or the second waist-crotch area have a thigh opening edge, respectively, the thigh opening edges extend along the anterior-posterior direction (X), each of the thigh opening edges is provided with a crotch portion elastic member enabling the thigh opening edges to be elastically contractible, and each of the thigh opening edges is provided with a second accommodating space formed along the anterior-posterior direction (X) for preventing the excrement from spilling out of the both sides of the crotch potion.

7. The diaper of claim 6, wherein the second waist portion are made of an elastically contractible nonwoven fabric.

8. The diaper of claim 7, wherein the nonwoven fabric of the second waist portion is joined to the second crotch end in a laminated manner, enabling the joined second crotch end and second waist-crotch area to form the first accommodating space.

9. The diaper of claim 6, wherein the joining of the second waist-crotch area and second crotch end is performed by a method selected from a group consisting of ultrasonic fusion joining and adhesive joining.

10. The diaper of claim 7, wherein the joining of the second waist-crotch area and second crotch end is performed by a method selected from a group consisting of ultrasonic fusion joining and adhesive joining.

11. The diaper of claim 8, wherein the joining of the second waist-crotch area and second crotch end is performed by a method selected from a group consisting of ultrasonic fusion joining and adhesive joining.
